(19) United States
(12) Patent Application Publication
(10) Pub. No.: US 2020/0121926 A1
(43) Pub. Date: Apr. 23, 2020

(54) SYSTEM AND METHOD OF MECHANICAL STIMULATION FOR PAIN RELIEF

(71) Applicant: Jennifer Jinus Tinoosh, Lynchburg, VA (US)

(72) Inventor: Jennifer Jinus Tinoosh, Lynchburg, VA (US)

(21) Appl. No.: 16/714,979

(22) Filed: Dec. 16, 2019

Related U.S. Application Data
(63) Continuation-in-part of application No. 14/326,349, filed on Jul. 8, 2014.
(60) Provisional application No. 61/843,704, filed on Jul. 8, 2013.

(51) Int. Cl. A61N 1/36; A61H 23/00; A61H 1/00; A61N 5/06; G16H 20/30

(52) U.S. Cl. CPC A61N 1/36021; A61H 23/006; A61H 1/008; A61N 2005/067; G16H 20/30; A61H 2201/10; A61N 5/0622

(57) ABSTRACT

System and method of mechanical nerve stimulation for pain relief via mechano-receptor treatment on inverse linked neuro-receptors of the spinal column is provided as an indirect means of treating localized pain through the administration of remote mechanical stimulation.

### TECHNICAL FIELD OF THE INVENTION

The invented method of treatment relates generally to the field of chiropractic treatment to alleviate symptoms of pain and increase range of motions using mechanical or photo stimulation of nerves for pain relief. This is a protocol that requires a device for the treatment.

### BACKGROUND OF THE INVENTION

Nerve stimulation has been used in the treatment of several chronic pain conditions including pain due to peripheral nerve dysfunctions, complex regional pain syndrome, and cranial neuralgias. Stimulation is done to areas of skin that are neurologically supplied from sensory nerve fibers from a single spinal nerve, or myotome, a group of muscles neurologically supplied by a single spinal nerve, or conventional anatomy such that when range of motion is limited in a joint due to pain or dysfunction, conventional chiropractic will treat the pain at/or around the site of pain.

Nerve receptors are activated by mechanical stimulation at either the site where spinal vertebrae interlock or they are activated at the joints of the limbs or peripherals. Many times, going to site of pain and mechanically manipulating the area, the patient will have adverse reactions to the treatment. As a patient may have reduced or increased sense of to touch or sensation, inflammation, hypersensitivity, or an increased sensitivity to pain, when you treat patient the defensive mechanism of the symptomatic area causes tension at the site, hence exerting manipulation or motion becomes challenging for the doctor and painful for the patient.

The present invention is directed to improved systems and methods for mechanical nerve stimulation for pain and other ailments.

### SUMMARY OF THE INVENTION

The present invention is directed to systems and methods of mechanical stimulation of an inversely linked neuro-receptor for pain relief. The pain or discomfort may be nerve or motor control related pain, an unspecified localized pain, joint site discomfort related pain, muscle or tendon condition related pain, or combinations thereof

In one embodiment, the method of mechanical stimulation of an inversely linked neuro-receptor for pain includes identifying a point of pain or discomfort on a patient's body, selecting an inversely linked neuro-receptor for the point of pain or discomfort, selecting the appropriate mechanical nerve stimulation device for the pain or discomfort, and administering the mechanical nerve stimulation inversely and/or contralaterally to the inversely linked neuro-receptor.

In some embodiments, the mechanical nerve stimulation device may be a high frequency percussive instrument, a high velocity-low amplitude instrument, a laser, an electrical stimulator, a TENS machine, or combinations thereof.

### BRIEF DESCRIPTIONS OF THE DRAWINGS

FIGS. 1 and 2 show a block diagram of a method of treatment for pain management using mechanical stimulation at inverse locations on the body.

FIGS. 3A and 3B is an anterior view and a posterior view displaying the inverse and contralateral arrangement of the point of pain and the point of mechanical stimulation treatment as per the current embodiment of the present invention.

FIG. 4 is an anterior view displaying the positioning of the dermatome regions on a human body.

FIG. 5 is a posterior view displaying the positioning of the dermatome region on the human body.

FIG. 6 is a frontal view displaying the separation of the superior region and the inferior region on the human body as per the current embodiment of the present invention.

FIG. 7 is a block diagram displaying the process overview of the method of pain management via mechano-receptor treatment on inverse linked neuro-receptors of the spinal column.

FIG. 8 is a block diagram displaying the process overview of the method of pain management via mechano-receptor treatment via mechanical stimulation of nerve or motor control related pain as well unspecific localized pain as per the current embodiment of the present invention.

FIG. 9 is a block diagram displaying the process overview of managing joint site related pain via mechanical stimulation as per the current embodiment of the present invention.

FIG. 10 is a block diagram displaying the process overview of managing muscle or tendon condition related pain via mechanical stimulation as per the current embodiment of the present invention.

The present invention is directed to improved systems and methods for mechanical nerve stimulation for pain and other ailments.

### DETAILED DESCRIPTION OF THE INVENTION

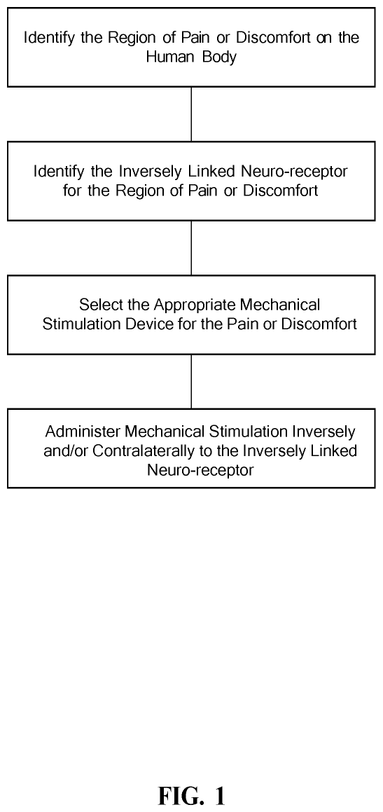

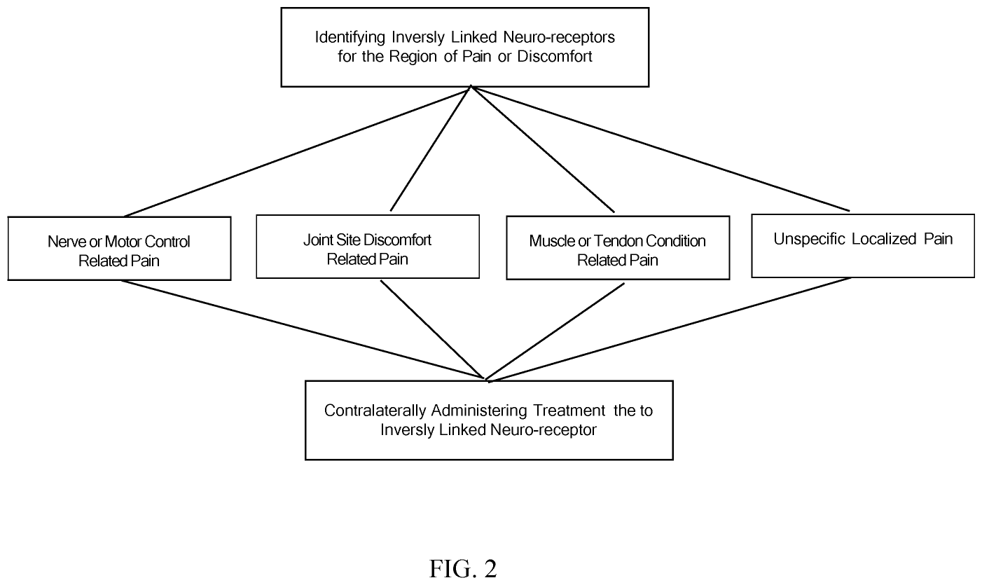

FIGS. 1 and 2 show a block diagram of a method of treatment for pain management using mechanical stimulation at inverse locations on the body, including: determination of a region of pain or discomfort; identifying the inversely linked neuro-receptor associated with the region of pain or discomfort; selection of the appropriate mechanical stimulator for the pain or discomfort; and application of mechanical stimulation inversely and/or contralaterally to the associated inversely linked neuro-receptor of the region of pain or discomfort.

The determination of a region of pain or discomfort can be accomplished through a plurality of means, such as a medical assessment or survey, to assess the type of pain or discomfort being experienced by the patient. The types of pain or discomfort may include: nerve or motor control related pain; joint site discomfort related pain; muscle or tendon condition related pain; or an unspecified localized pain. Interactions with the patient may also be used for locating the particular region of pain or discomfort and as a result would enable a practitioner to then assess the type of pain or discomfort being experienced by the patient.

Identifying the inversely linked neuro-receptor associated with the region of pain or discomfort these locations may include; inversely paired dermatomes, inversely paired joint sites; or inversely paired muscle or tendon sites. The treatment administers mechanical stimulation inversely and/or contralaterally to the region of pain or discomfort. Inversely paired dermatomes are disclosed in U.S. patent application Ser. No. 14/326,349, each of which is commonly owned with this application and each of which is herein incorporated by reference.

The type of pain or discomfort is used to determine the appropriate mechanical stimulation device to use. The mechanical stimulation may be specialized equipment for nerve stimulation, such as: a high frequency percussive instrument, a high velocity-low amplitude instrument, a laser; an electrical stimulator. The high frequency percussive instrument may deliver waves of percussive impulses deep into the tissues of the body, which promote an increase in circulation. The pulsation tool may deliver 12-14 incremental thrusts per second, making it much stronger and faster than a typical single thrust administered by a chiropractor. The laser may use laser light to penetrate tissue. The electrical stimulator may be a TENS machine that delivers small electrical impulses through electrodes that have adhesive pads to attach them to a skin.

In one embodiment of the invention, the mechanical stimulation inversely and contralaterally to the region of pain or discomfort, which may be done to inversely paired dermatomes.

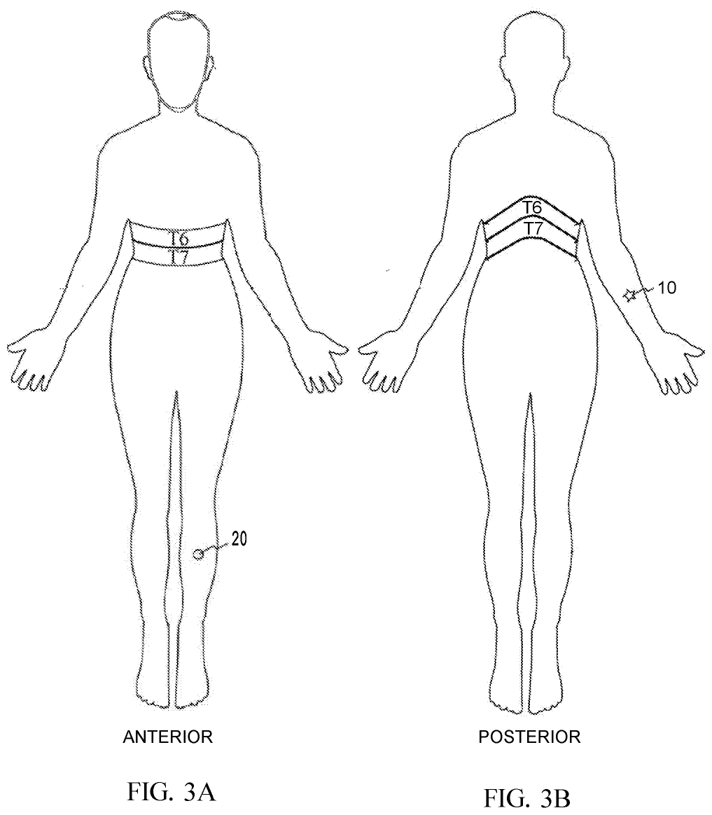

FIGS. 3A and 3B is an anterior view and a posterior view displaying the inverse and contralateral arrangement of the point of pain and the point of treatment via mechanical stimulation providing. A region of pain or discomfort 10 is identified on the superior region of the inferior region. An inversely linked neuro-receptor 20 is identified for a treatment location. A mechanical stimulator 30 is selected based on the pain or discomfort. Mechano-stimulation is then applied to the inversely linked neuro-receptor 20.

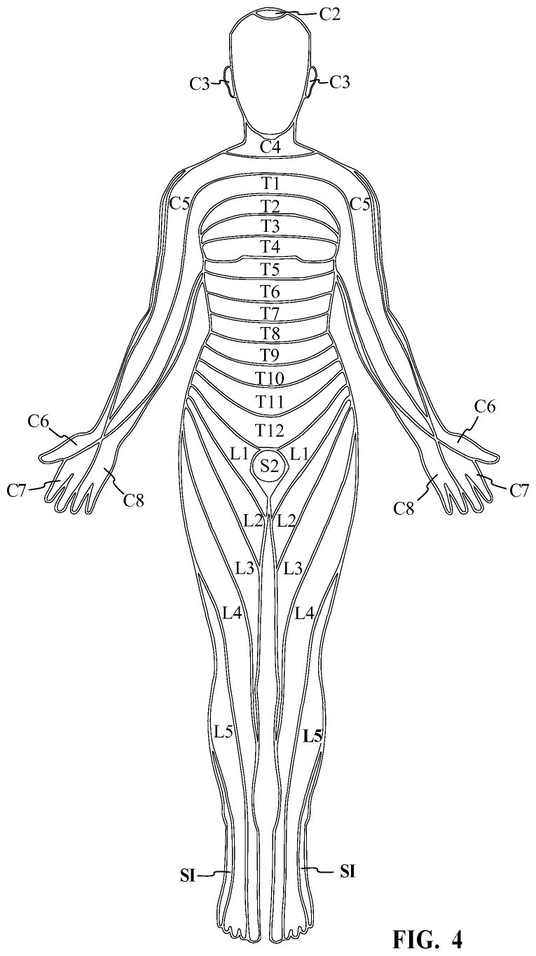

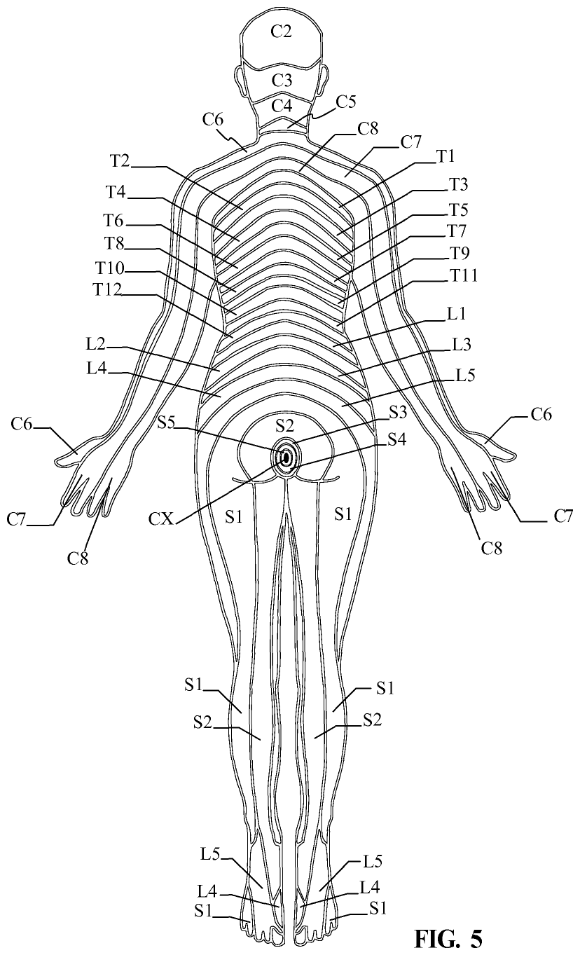

FIG. 4 and FIG. 5 show dermatome regions on the patient's body that are known to relate to particular nerve ending extending from the spinal cord.

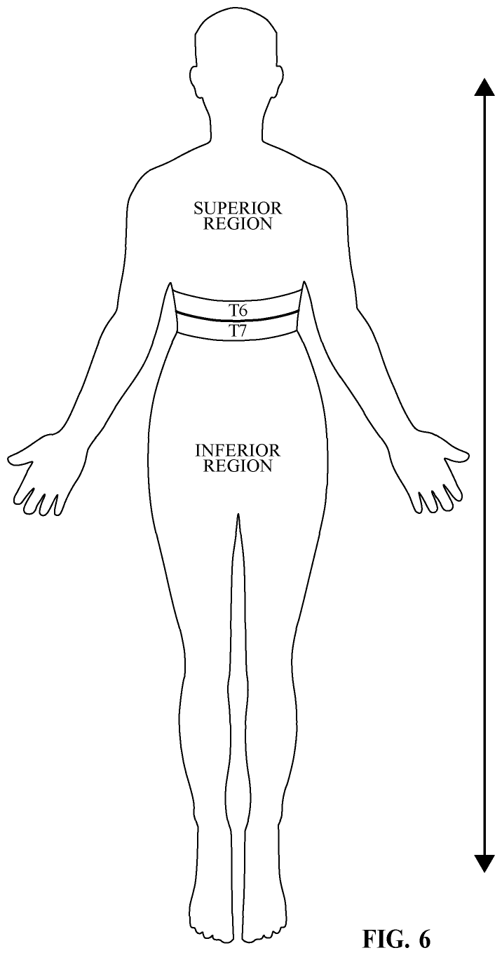

Additionally the human body is further divided in a superior region and an inferior region along the superior-inferior axis. Referencing FIG. 6, the superior region and the inferior region are separated by the coincident edge of the T6 dermatome region with the T7 dermatome region. The superior region extends from the cranium towards the coincident edge of the T6 region, while the inferior region extends from the coincident edge of the T7 region towards the patient's feet. The division of the superior region and the inferior region provides distinct contralaterally positioned points of treatment based on the identified points of pain. The utilization of the dermatome regions permits a direct reference for identifying a point of pain 10 and determining its corresponding point of treatment 20.

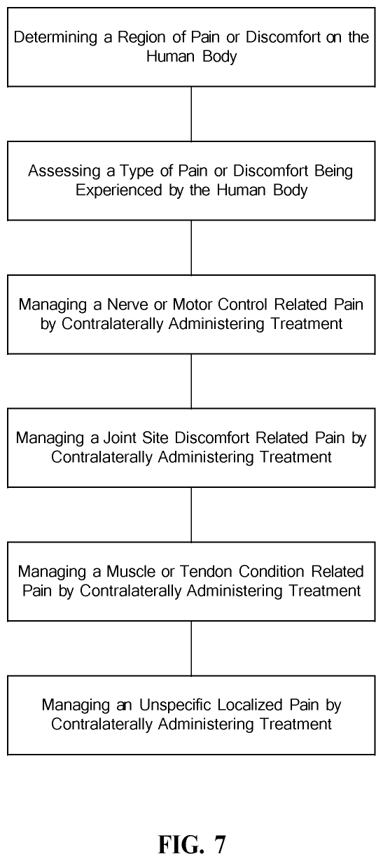

FIG. 7 is a block diagram displaying the process overview of the method of pain management via mechano-receptor treatment on inverse linked neuro-receptors of the spinal column In some embodiments, the method of pain management via mechano-receptor treatment of inverse linked neuro-receptors of the spinal column comprises the steps of determining a region of pain or discomfort on the human body, assessing the type of pain or discomfort being experienced by the human body, and managing the pain on the superior region or the inferior region by contralaterally administering treatment on the inferior region or the superior region respectively. The determination of the region of pain or discomfort on the human body locates the particular region to which the pain or discomfort is associated with. The determination of the particular region of pain or discomfort is utilized in conjunction with the assessment of the particular type of pain or discomfort in order to determine a corresponding point of treatment 20 on the human body. The assessment of the type of pain is performed to determine if the patient is suffering from nerve or motor control related pain, joint site discomfort related pain, a muscle or tendon condition related pain, or an unspecified localized pain. The management of the pain is accomplished through the administration of treatment to an inverse and contralaterally positioned point of treatment 20 relative to the region of pain and to the type of pain or discomfort experienced at the point of pain 10.

The determination of a region of pain or discomfort is accomplished through interactions with the patient. The interaction with the patient would provide vital information for locating the region of pain or discomfort and as a result would enable a practitioner to then assess the type of pain or discomfort being experienced by the patient. The interactions with the patient may be accomplished through a medical assessment or survey, wherein the patient would provide the information regarding the point of pain 10 or discomfort as part of the medical assessment. It should be understood that the determination of the region of the pain and the action that causes the pain or discomfort can be accomplished through a plurality of means and that the importance of the step is for the localizing the region of pain or discomfort in order to later assess the type of pain being experienced by the patient.

The assessment of the type of pain and the action that causes the pain being experienced by the patient is utilized in conjunction with the determination of the region of pain or discomfort to implement a treatment to a point of treatment 20. The assessment step makes the determination between four categories of pain which are location and interaction dependent. The four categories of pain comprise nerve or motor control related pain, joint site discomfort related pain, muscle or tendon condition related pain, and unspecific localized pain or a combination of the aforementioned. Nerve or motor control related pain is pain associated with particular nerve endings that respond painfully to particular motions or actions. Nerve or motor control related pain is assessed by a practitioner through a plurality of assessment interaction that include but are not limited to palpation of and/or stimulation of the region of pain or discomfort. Due to the particular nature of the nerve or motor control related pain, the point of pain 10 and the point of treatment 20 are closely associated with dermatome regions, muscle functions and contralateral joints from upper body to the lower body. As a result of this relation, regions of pain or discomfort caused by nerve or motor control related pains are directly correlated with dermatome regions or muscle functions and contralateral joints from upper body to the lower body. Similar to nerve or motor control related pain, unspecific localized pains such as non-muscular or joint related pains would also rely on contralateral joints from the upper body to the lower body, front of the thorax to the back of the thorax regions. Unspecific localized pain can include headaches and non-muscular abdominal aches and pains. Joint site discomfort related pain is related to pain felt by a patient at a particular joint site during movement or following movement of the particular joint site. Joint site discomfort related pain does not rely on dermatome regions as many joint sites have comparable inversely and contra laterally positioned joint site that function as a point of treatment 20. Muscle or tendon condition related pains are pains related to particular muscle groups, connective tissue, or tendons as a result of an injury or motion. Muscle or tendon condition related pain do not specifically rely on dermatome regions for localization of point of pain 10 and point of treatment 20 although in many instances the point of pain 10 and point of treatments 20 will overlap with dermatome regions.

TABLE 1
Treatment Nerve Root Pairing for
Management of Nerve or Motor Control Related Pain

| Point of pain | Point of treatment |
|---|---|
| C1/Occiput | COCCYX |
| C2 | SACROILIAC NERVE |
| C3 | L5 |
| C4 | L4 |
| C5 | L3 |
| C6 | L2 |
| C7 | L1 |
| T1 | T12 |
| T2 | T11 |
| T3 | TI0 |
| T4 | T9 |
| T5 | T8 |
| T6 | T7 |
| T7 | T6 |
| T8 | T5 |
| T9 | T4 |
| TI0 | T3 |
| T11 | T2 |
| T12 | T1 |
| L1 | C7 |
| L2 | C6 |
| L3 | C5 |
| L4 | C4 |
| L5 | C3 |
| SI Sacroiliac Nerve | C2 |
| COCCYX | C1/Occiput |

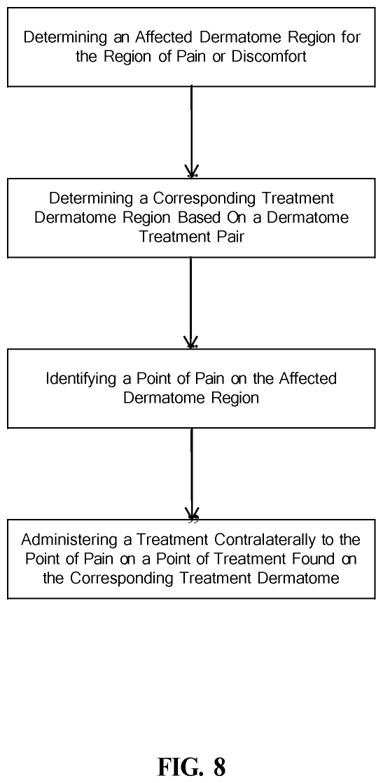

TABLE 1 shows the inverse linked nerve roots related to nerve or motor control. FIG. 8 is a block diagram displaying the process overview of nerve or motor control related pain as well unspecific localized pain.

The treatment of neuro-receptors of nerve or motor control related pain is accomplished through inverse administration of treatment contralaterally on an opposing region on the superior-inferior axis. A point of pain 10 assessed as a nerve or motor control related pain is identified as being on either the superior region of the inferior region, at which point the point of treatment 20 is determined to be positioned contralaterally on the inverse region of the point of treatment 20, either the inferior region or the superior region, respectively. The management of nerve or motor control related pain is dependent on the determination of an affected dermatome region or nerve root. If the pain is muscular then the muscles are evaluating contralaterally superior to inferior the region of pain or discomfort experienced by the patient. The practitioner would determine the affected dermatome region by assessing the region of pain or discomfort and correlate the findings with the known distribution and positioning of dermatome regions. Upon determining an affected dermatome region, the practitioner would be able to determine a corresponding treatment dermatome region based on a dermatome treatment pair for nerve or motor control related pain. The dermatome treatment pair for nerve or motor control related pain is a relationship between dermatome regions positioned opposite the separation of the superior region and the inferior region. The dermatome treatment pair comprises a dermatome in the superior region and a dermatome in the inferior region. The understanding is provided that the treatment is administered on the opposing nerve roots as it is shown on the chart. The opposing dermatomes/Nerve roots are related and pain or discomfort to either region would be treatable through administration of a mechano-stimulation/or photo via class IV cold laser stimulation to the other paired dermatome/nerve root. Following the identification of the corresponding treatment dermatome/Nerve root region, a point of pain 10 would be identified on the affected dermatome/Nerve root region. The point of pain 10 is found through palpation or through interactions with the patient. The identification of the point of pain 10 permits the identification of the point of treatment 20 on the corresponding treatment dermatome. The point of treatment 20 is contralaterally positioned to the point of pain 10 on the treatment dermatome. The administration of a treatment would be conducted by the practitioner to the point of treatment 20. Following administration, the practitioner would verify the effectiveness of the treatment.

It should be noted that the treatment dermatome pairing comprising C1 and the COCCYX is provided with an understanding that the C1 cervical nerve has no associated dermatome region. C1 is unique as the C1 root innervates the meninges of the posterior fossa. A determination of the point of pain 10 being at C1 is particular to nerve or motor control related pain. As a result assessment of the type of pain is greatly important to ensure that proper administration of a treatment to a particular point of treatment 20. When determined as the point of treatment 20, treatment is administered to C1 nerve through the C2 dermatome region.

TABLE 2
Treatment Dermatome Pair for
Management of an Unspecific Localized Pain

| Point of pain | Point of treatment |
|---|---|
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |
| Acetabular Joint | Gleno-Humeral Joint |
| Anterior Iliac Spine | Acromioclavicular Joint |
| Posterior Superior Iliac Spine | Sterno-Clavicular Joint |
| Fibulo-Femoral Joint | Ulnar-Humeral Joint |
| Tibio-Femoral Joint | Radio-Humeral Joint |
| Inferior Patella | Olecranon Process |
| Tibio-Fibular Joint | Radio-Ulnar Joint |
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |

TABLE 2 shows the inverse linked neuro-receptors related to unspecified localized pain. The treatment contralaterally on an opposing region on the superior-inferior axis. Unspecific localized pain differs from the other categories of pain as it is related specifically to an area of skin or to a dermal region associated with a dermatome. The management of unspecific localized pain is approached similarly to the treatment of nerve or motor control related pain. Similarly, the unspecific localized pain is identified as being associated with a point of pain 10 on an affected dermatome region on either the superior region of the inferior region. The identification of the point of pain 10 permits the identification of a point of treatment 20 positioned contralaterally on the inverse region being either the inferior region or the superior region, respectively. Similar to the management of nerve or motor control related pain, management of unspecific localized pain is dependent on the determination of an affected dermatome region. The affected dermatome region is the affected dermatome on which the region of pain or discomfort is being experienced by the patient. A practitioner would determine the affected dermatome/nerve root/muscles or joints region by assessing the complaints of the patient and correlating the region of pain or discomfort with the known distribution and positioning of dermatome dermatome/nerve root/muscles or joints regions. Upon determining an affected dermatome dermatome/nerve root/muscles or joints region, the practitioner would be able to determine a corresponding treatment dermatome region based on a dermatome treatment pair for unspecific localized pain. The dermatome treatment pair for the unspecific localized pain is a relationship between dermatome regions positioned opposite the separation of the superior region and the inferior region. The dermatome treatment pair comprises a dermatome in the superior region and a dermatome in the inferior region. The understanding is provided that the opposing dermatomes are related and pain or discomfort to either region would be treatable through administration of a mechano-stimulation/Photo stimulation(class IV cold laser) to the other paired dermatome. Following the identification of the corresponding treatment dermatome dermatome/nerve root/muscles or joints region, a point of pain 10 would be identified on the affected dermatome dermatome/nerve root/muscles or joints region. The point of pain 10 is found through palpation or through interactions with the patient. The identification of the point of pain 10 permits the identification of the point of treatment 20 on the corresponding treatment dermatome dermatome/nerve root/muscles or joints. The point of treatment 20 is contralaterally positioned to the point of pain 10 on the treatment dermatome dermatome/nerve root/muscles or joints. The administration of a treatment would be conducted by the practitioner to the point of treatment 20. Following administration, the practitioner would verify the effectiveness of the treatment.

It should be noted that the treatment dermatome pairs for the management of unspecific localized pain differs from the treatment dermatome pairs for the management of the nerve or motor control related pain at the dermatome site of the Coccyx. The treatment dermatome pair for the management of the nerve or motor control related pain is nerve specific while dermatome regions relate to dermal regions associated with the particular nerve ending. Resultantly, the dermatome treatment pair for the coccyx would be accomplished by the C1 nerve root in the nerve or motor control treatment pair. In the unspecific localized pain treatment pair, the coccyx corresponds to the C2 nerve root region causing a shift for the treatment pairs. As a result, C3 is paired with the SI Sacroiliac Nerve roots exiting from the sacrum, C4 is paired with L5, C5 is paired with L4, C6 is paired with L3, C7 is paired with L2, and C8 is paired with L1.

TABLE 3
Joint Site Treatment Pair for
Managing Joint Site Discomfort Related Pain

| Point of pain | Point of treatment |
|---|---|
| Gleno-Humeral Joint | Acetabular Joint |
| Stemo-Clavicular Joint | Posterior Superior Illiac Spine |
| Ulnar-Humeral Joint | Fibulo-Femoral Joint |
| Radio-Humeral Joint | Tibia-Femoral Joint |
| Olcranon Process | Inferior Patella |
| Radio-Ulnar Joint | Tibia-Fibular Joint |
| Radial Site | Medial-Malleolus |
| Ulnar Site | Lateral-Malleolus |
| Scaphoid | Navicular |
| Lunate | Medial-Calcaneus |
| Triquetrum | Lateral Calcaneal |
| Hamate | Lateral Cuneiform |
| Trapezium | Intermedial Cuneiform |
| Trapezoid | Medial Cuneiform |
| Capitate | Cuboid |
| Meta-Carpo-Carpal Joint | Meta-Tarsal Joint |
| Meta-Carpo-Phalangeal | Meta-Tarso-Halux Phalangeal |
| Policis | Halux |
| Proximal Phalangeal | Pedal Proximal Phalangeal |

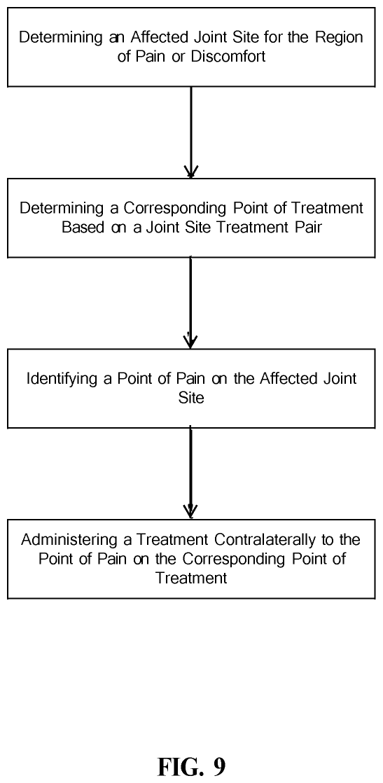

TABLE 3 shows the inverse linked joint treatment site related to joint site pain. FIG. 9 is a block diagram displaying the process overview of managing joint site related pain.

The treatment is accomplished utilizing the same principal as the nerve or motor control related pain and the unspecific localized pain, in that an affected region is remotely treated contralaterally and on an opposing region, relative to the superior region and inferior region. Unlike the management of the nerve or motor control related pain and the unspecific localized pain, the management of the joint site discomfort related pain does not rely on dermatome regions to identify a region of pain or discomfort. The management of the joint site discomfort related pain identifies particular joint sites as joint site treatment pairs. The joint site treatment pairs comprise an affected joint site and a treatment joint site. The joint site treatment pairs are positioned contralaterally and on opposing regions, either the superior region or the inferior region.

The management of the joint site discomfort related pain is accomplished through the determination of an affected joint site as the region of pain or discomfort. An affected joint site is identified by a practitioner through a medical assessment that may include palpation of the region of pain or discomfort. The practitioner can additionally request the patient articulate the particular joint to make the determination. Upon the determination of the affected joint site as the region of pain or discomfort, the practitioner identifies a point of pain 10 on the affected joint site. The identification of the point of pain 10 provides specificity that helps in the administration of the treatment. Following the identification of the point of pain 10, the practitioner determines a corresponding point of treatment 20 to the point of pain 10 based on a joint site treatment pair. The joint site treatment pair allows a practitioner to determine the corresponding treatment joint site. The corresponding treatment joint site is utilized with the knowledge of the point of pain 10 to correctly identify the contralateral positioning of the point of treatment 20 on the corresponding treatment joint site. The practitioner administers mechanical stimulation to the treatment joint site as necessary. Following the administration of the mechanical stimulation, the practitioner reevaluates the patient to determine the effectiveness of the treatment.

It should be noted that the joint site treatment pairs comprise the Meta-Carpo-Carpal Joint paired with the Meta-Tarsal Joint, the Meta-Carpo-Phalangeal paired with the Meta-Tarso-Halux Phalangeal, the Proximal Phalangeal paired with the Pedal Proximal Phalangeal, and the Distal Phalangeal paired with the Distal Pedal Phalangeal. The aforementioned joint site treatment pairs are each related to a particular digit of the users hand or foot. It should be understood that the joint pairing would correspond to the particular joint on the corresponding digit of the patients hand or foot such that the affected joint site on the Meta-Carpo-Carpal Joint of the first digit on the patients hand would correspond with the Meta-Tarsal Joint of the first digit on the patient's foot.

In the current embodiment of the present invention, the management of joint site discomfort is provided with a join site treatment triplet for particular joint sites. It has been observed that the particular administration of mechanical stimulation to two remote treatment joint sites for a particular affected joint site has achieved favorable treatment outcomes. The particular joint sites in question comprise the Temporomandibular Joint (TMJ), the Hip Joint, and the shoulder. A particular relationship has been determined between the temporomandibular joint and the shoulder, where treatment administration on either is ipsilateral to one another. For instance, an identification of the point of pain 10 on the right temporomandibular joint would result in an ipsilater point of treatment 20 on the right shoulder, but a contralateral point of treatment 20 on the hip joint. Furthermore, in another example, the identification of the point of pain 10 on the right hip joint would result in a contralateral point of treatment 20 for both the shoulder and the temporomandibular joint.

In the current embodiment of the present invention, the management of joint site discomfort related pain additionally comprises the directional treatment administration to a treatment joint site. Directional treatment administration is provided to particular join site treatment pair comprising the acromioclavicular joint and the anterior iliac spine. The direction treatment administration is provided when the point of pain 10 is determined on the acromiclavicular joint. It has been determined that the administration treatment to the anterior iliac spine produces the most favorable results when a mechanical stimulation is administered to the anterior iliac spine from the anterior superior iliac spine towards the anterior inferior iliac spine.

TABLE 4
Muscle or Tendon Treatment Pair for
Managing a Muscle or Tendon Condition Related Pain

| Point of pain | Point of treatment |
|---|---|
| Knee Flexors | Elbow Flexors |
| Knee Extensors | Elbow Extensors |
| Knee Internal Rotators | Elbow External Rotators |
| Knee External Rotators | Elbow Internal Rotators |
| Wrist Flexors | Ankle Plantar Flexors |
| Wrist Extensors | Ankle Dorsiflexors |
| Wrist Medial Flexors | Ankle Pronators |
| Wrist Lateral Flexors | Ankle Supinators |
| Shoulder Flexors | Hip Extensors |
| Shoulder Extensors | Hip Flexors |
| Shoulder Abductors | Hip Abductors |
| Shoulder Adductors | Hip Adductors |
| Shoulder Internal Rotators | Hip Extrnal Rotators |
| Shoulder External Rotators | Hip External Rotators |
| Neck Flexors | Lumbar Flexors |
| Neck Extensors | Lumbar Extensors |
| Cervical Rotators (SCM) | Ipsilateral Illiopsoa Muscle Insertion |
| Supra Spinatus | Gluteus Minimus/Subclavicus |

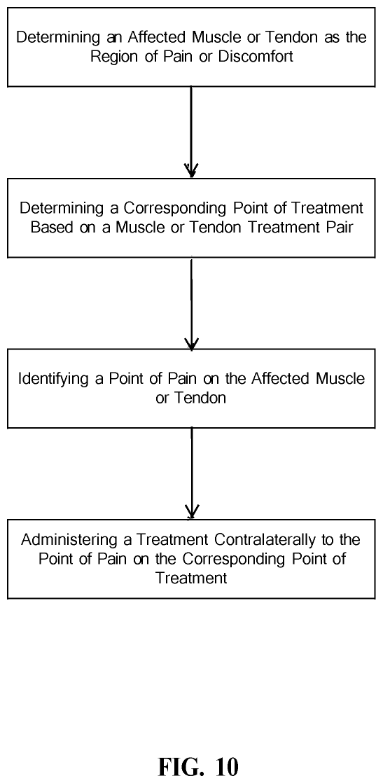

TABLE 4 shows the inverse linked muscle or tendon treatment site related to muscle or tendon condition related pain. FIG. 10 is a block diagram displaying the process overview of managing muscle or tendon condition related pain.

The treatment is provided as a means of treating pains related to particular muscle groups, connective tissue, or tendons as a result of an injury or particular range of motion. Management of muscle or tendon condition related pain is accomplished through remote administration of treatment on the contralateral and inverse location to the region of pain or discomfort on a patient's body. Due to the points of pain and the points of treatment being derived from muscle or tendon conditions, the management of pain is greatly dependent on an understanding of the skelatol-muscular system. When the patient hurts with certain muscle or joint action we treat the patient at the site of treatment while the patient is mimicking those movements until the symptoms are significantly dissipated or completely diminished. The treatment pair for the muscle or tendon condition related pains is based on muscle or tendon sites that are inverse and contralaterally positioned to one another. Additionally, an affected muscle or tendon site can be activated and treated by a particular range of motion. A practitioner determines an affected muscle or tendon as the region of pain or discomfort through a medical assessment that can include palpation to the muscle or tendon as well as requesting the patient to move in a particular manner to illicit the particular pain response. Following the determination of the affected muscle or tendon, the practitioner would identify the particular point of pain 10 on the affected muscle or tendon. The identification of the point of pain 10 is necessary as the means of determining the particular point of treatment 20. The particular point of treatment 20 is determined through a corresponding muscle or tendon treatment pair. The muscle or tendon treatment pair comprises an affected muscle or tendon as well as a corresponding treatment muscle or tendon. The muscle or tendon treatment pairs are contralaterally and inversed positioned to one another. When an affected muscle or tendon is found on an extremity, the corresponding treatment muscle or tendon is found contralateral and an opposing limb. Following the identification of the corresponding treatment muscle or tendon, the practitioner administers the mechanical stimulation to the treatment muscle or tendon on the corresponding point of treatment 20. After the treatment is administered, the practitioner reevaluates the patient to ensure the effectiveness of the treatment.

In the current embodiment of the present invention, the point of pain 10 on an affected muscle or tendon may be directionally activated with a particular bias in movement. The particular bias in the direction of activation for the affected muscle or tendon would be such that a clockwise rotation would activate the point of pain 10. In the aforementioned example, administration of a counter directional movement on the point of treatment 20 would be deemed appropriate on the corresponding treatment muscle or tendon associated with the affected muscle or tendon on which the point of pain 10 is found on. In an embodiment of the present invention, generalized neck pain upon rotation in cervical paraspinal is alleviated through the rotation of the lumbar to the opposite side and treating the lumbar paraspinalis. Similarly, the treatment of a cervical paraspinal pain upon lateral (right or left) bending is treated through a bend at the lumbar spine to the opposite direction of the cervical lateral bending that causes the pain and would include mechanical stimulation/manipulation on the lumbar paraspinal to the side of bending in order to alleviate pain.

In the current embodiment of the present invention, headaches are treatable through complimentary locations on the navel region if the headache is frontal, however occipital headaches are treat by the stimulation of the tip of xyphoid process or sacrococcygeal area. Frontal headache are treatable through the administration of treatment to a region one inch above the naval and one inch lateral each way going across. Coronal headaches are treatable through the administration of treatment to the sacro coccyxgeal area. Furthermore, parietal headaches are treatable through the administration of contralateral stimulation to the gluto-acetabular region.

The treatment of heel pain (heel spur inflammation or plantar fasciitis) is administered at the contralateral thenar level adjacent to the radial head if the pain is medial calcaneal pain if it is lateral calcaneal pain it is on hypothenar adjacent or over the pisiform region.

The chiropractic Protocol method for pain management is able to remotely treat an affected area of a patient's body. The preset invention accomplishes this through a method for administrating mechanical stimulation/photo stimulation at a position that is diagonal from the affected region. The remote administration of mechanical stimulation/phot stimulation relieves pain caused by a plurality of reasons and through the understanding of the theory of four forces, assure zero pressure at the center of force, resulting in restored balanced posture and a full range of motion. In the current embodiment of the present invention, the method utilizes dermatome regions/nerve roots, myotomes, contralateral biomechanics in order to distinguish the pain regions as well as subsequent treatment regions. It should be noted that while dermatome regions regions/nerve roots, myotomes, contralateral biomechanics are utilized in the present invention, the current embodiment utilizes a novel interpretation of the dermatome regions regions/nerve roots, myotomes, contralateral biomechanics that provides facilitated identification of pain/discomfort areas as well as contra-laterally treatment regions. Furthermore, it should be noted that treatment regions are dependent on the type of pain that is experienced by the user.

The treatment method comprises the steps of determining the region of pain or discomfort, assessing the patient's type of discomfort, determining contra-laterally positioned treatment region on the patient's body, administering the treatment to the patient, and reevaluating the patient's condition. The determination of the region of pain or discomfort is provided as the step of the method that associates a patient's discomfort with a dermatome region regions/nerve roots, myotomes, contralateral biomechanics. The assessment of the patient's pain is provided as the step of the method that determines the type of pain/discomfort the patient is feeling. The determination of the contra-lateral positioning of the treatment region on the patient's body is provided as an identifying step for determining the contra-lateral position of treatment administration based on the type of pain and the associated pain region. The administration of the treatment to the patient is provided as the step of the method that mechanical stimulation would administer to the determined treatment region. The revaluation of the patient is provided as the step of the method that assesses the success of the treatment. Through the combination of the aforementioned method steps, the treatment method would provide an alternative pain management treatment.

The method step of determining the region of pain or discomfort for a patient is the step that is utilized to locate the region that the patient is experiencing discomfort. During the step, a practitioner would assess the patient's general complaint and utilize a dermatome diagram regions/nerve roots, myotomes, contralateral biomechanics and their anatomical knowledge to make a determination regarding the patient's specific area of discomfort. Additionally, the practitioner would utilize their knowledge to make determination regarding joint sites as well as muscle and tendon site to administer treatments. It should be noted that compound injuries and a combination of various injuries would be considered, and a practitioner may utilizes their discretion to determine on which region of discomfort to focus on first.

The method step of assessing the patient's type of discomfort is the step that is utilized to determine if the patient is experiencing pain/discomfort that is related with nerve and motor control issues or if the patient is experiencing discomfort due to a particular dermatome. During the step, the practitioner would utilize the patient's medical history and exam the patient's region of discomfort to make the determination regarding what type of pain the patient is feeling. It should be noted that the determination step for locating the patient's region of discomfort is a prerequisite step for evaluating the patient's pain type, as the type of pain may be essential in determining the corresponding treatment region for the patient's particular pain.

The method step of determining the contra-laterally positioned treatment region on the patient's body is provided as the step where the practitioner utilizes the prior steps and their knowledge to locate the treatment region based on Table 1-4. During the step, the practitioner would evaluate the region of discomfort and the pain type and reference Table 1 and Table 2 to determine the contra laterally positioned treatment location. Additionally, the practitioner would utilize Table 3 and Table 4 to make determinations regarding discomfort felt in particular joint sites and muscle and tendon regions.

The method step of administering the treatment to the patient is provided as the step where the practitioner administers mechanical stimulation or photo stimulation via cold class IV laser to the determined treatment location. During the step the practitioner would administer mechanical stimulation as need to the pre-determined treatment location wherein the duration and intensity of the mechanical stimulation would be dependent of the intensity and type of discomfort felt by the patient. It should be noted that the duration and intensity of the mechanical stimulation would be influenced by the practitioner's knowledge of the patient's particular condition and could deviate as needed in order to accommodate the patient's needs.

The method step of reevaluating the patient's condition is provided as the step where the practitioner determines the success of the treatment and the further determines a treatment schedule for the patient. During the step the practitioner evaluates the treatment's effectiveness by determining changes to the patient's initial complaints; results are normally seen immediately after administering the treatment. Typically the results will show a 40-50% improvement in range of motion complaints and a noticeable difference in dermatome, joint, and muscle and tendon pain. It should be noted that the practitioner may additionally recommend follow up visits as needed at this point.

The sagittal plane is utilized in order to function as a dividing plane that separates the right and left side of the body. The division provides the treatment method with a mid-line in order to establish diagonally positioned points from right to left, left to right, top to bottom, and bottom to top. In accordance with the current embodiment of the present invention, discomfort felt in the upper thorax, or the area between the neck and diaphragm which is incased by the ribs and mid-spine, is treated by the lower thorax. Similarly, the cervical-occipital region, or neck area, is treated by the lumbo-sacro-coccygeal region or the lower region of the spine-triangular bone at the end of the spine. Additionally, the cranium region is treated by the lower gluteal or abdominal region and the upper extremities are treated by the lower extremities and vice versa. Adhesive capsulitis or frozen shoulder is treated by administering the treatment on the contralateral hip joints, calf muscles and the hamstring group as well as the gluteal area since the rotator cuff is a complex muscle joint area.

The treatment method locates site of pain from patient, as well as what aggravates or amplifies the pain. Once point of pain 10 and amplifiers are determined, the treatment method stimulates the neuro-mechano receptors using a chiropractic instrument or laser class IV cold laser used to treat joint, nerve, and muscular systems via repetitive pulsations. At the opposite point of the sagittal plane as described above. An example of this situation would be if a patient is complaining of pain within the upper extremity region, the treatment method would stimulate the neuro-mechano receptors at the opposite plane point within the lower extremity region on the opposite side of the body.

Within spinal articular fixation or lesions, which is a mechanical dysfunction within the spinal joints, the treatment method would focus on the zygapophyseal joints at the sister vertebrae to treat mechanical derangement. For neurological disorders such as numbness or tingling known as paresthesia or weak muscles due to poor innervation, the treatment method would be administered at the sister nerve root to balance the axoplasmic flow of the nerve pattern, which is basically the circulation of fluid between the cell body of a neuron to the tail of the same neuron, known as the axon or the terminal process of a neuron.

In the current embodiment of the present invention, the treatment method activates the muscle spindle (stretch receptor) by stretching the muscle on the opposite plane within the opposite extremity region. By stretching the opposite plane muscle within the opposite extremity region, the stretched muscle communicates signals to the injured muscle spindle that increases range of motion and decreases pain. A similar phenomenon applies at the spinal level between the sister vertebral joints via stimulating the proprioceptors, which are nerve receptors associated with movement and position of the body. When treatment is administered to the sister vertebrae, the misaligned vertebrae regains increased range of motion and the patient experiences pain relief, restored joint function and a better range of motion.

Although the invention has been explained in relation to its preferred embodiment, it is to be understood that many other possible modifications and variations can be made without departing from the spirit and scope of the invention as hereinafter claimed.

Considering the fact that when a patient is in so much pain they don't want the area of pain to be manipulated pulled or pushed, this invented method/protocol gives the practitioner the liberty to treat the patient more effectively in much shorter period of time without insulting the area of pain. Also, considering the use of opioids and the crisis the invented method allows the patients who are apprehensive to see a conventional chiropractor feel more comfortable having the treatment to be done remotely without touching the inflamed point or region of discomfort area.

What is claimed is:

1. A method of mechanical stimulation of an inversely linked neuro-receptor for pain relief comprising:
identify a point of pain or discomfort on a patient's body;
selecting an inversely linked neuro-receptor for the point of pain or discomfort;
select the appropriate mechanical nerve stimulation device for the pain or discomfort; and
administer mechanical nerve stimulation inversely and/or contralaterally to the inversely linked neuro-receptor.

2. The method of claim 1, wherein the mechanical nerve stimulation device is selected from the group consisting of: a high frequency percussive instrument, a high velocity-low amplitude instrument, a laser; an electrical stimulator, a TENS machine, and combinations thereof.

3. The method of claim 1, wherein the pain or discomfort is selected from the group consisting of: nerve or motor control related pain; an unspecified localized pain; joint site discomfort related pain; muscle or tendon condition related pain; and combinations thereof.

4. The method of claim 3, wherein the pain or discomfort is nerve or motor control related pain and the inversely linked neuro-receptor is an inversely linked dermatome selected from the group consisting of:

| Point of pain Dermatome | Point of treatment Dermatome |
|---|---|
| C1 | COCCYX |
| C2 | SI SACROILIAC NERVE |
| C3 | L5 |
| C4 | L4 |
| C5 | L3 |
| C6 | L2 |
| C7 | L1 |
| T1 | T12 |
| T2 | T11 |
| T3 | T10 |
| T4 | T9 |
| T5 | T8 |
| T6 | T7 |
| T7 | T6 |
| T8 | T5 |
| T9 | T4 |
| T10 | T3 |
| T11 | T2 |
| T12 | T1 |
| L1 | C7 |
| L2 | C6 |
| L3 | C5 |
| L4 | C4 |
| L5 | C3 |
| SI Sacroiliac Nerve | C2 |
| COCCYX Phalangeal | C1. |

5. The method of claim 3, wherein the pain or discomfort is an unspecified localized pain, and the inversely linked neuro-receptor is an inversely linked dermatome selected from the group consisting of:

| Point of pain Dermatome | Point of treatment Dermatome |
|---|---|
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |
| Acetabular Joint | Gleno-Humeral Joint |
| Anterior Iliac Spine | Acromioclavicular Joint |
| Posterior Superior Iliac Spine | Sterno-Clavicular Joint |
| Fibulo-Femoral Joint | Ulnar-Humeral Joint |
| Tibio-Femoral Joint | Radio-Humeral Joint |
| Inferior Patella | Olecranon Process |
| Tibio-Fibular Joint | Radio-Ulnar Joint |
| Point of pain | Point of treatment |
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |

6. The method of claim 3, wherein the pain or discomfort is joint site discomfort related pain and the inversely linked neuro-receptor is selected from the group consisting of:

| Point of pain Dermatome | Point of treatment Dermatome |
|---|---|
| Gleno-Humeral Joint | Acetabular Joint |
| Stemo-Clavicular Joint | Posterior Superior Illiac Spine |
| Ulnar-Humeral Joint | Fibulo-Femoral Joint |
| Radio-Humeral Joint | Tibia-Femoral Joint |
| Olcranon Process | Inferior Patella |
| Radio-Ulnar Joint | Tibia-Fibular Joint |
| Radial Site | Medial-Malleolus |
| Ulnar Site | Lateral-Malleolus |
| Scaphoid | Navicular |
| Lunate | Medial-Calcaneus |
| Triquetrum | Lateral Calcaneal |
| Hamate | Lateral Cuneiform |
| Trapezium | Intermedial Cuneiform |
| Trapezoid | Medial Cuneiform |
| Capitate | Cuboid |
| Meta-Carpo-Carpal Joint | Meta-Tarsal Joint |
| Meta-Carpo-Phalangeal | Meta-Tarso-Halux Phalangeal |
| Policis | Halux |
| Proximal Phalangeal | Pedal Proximal Phalangeal |

7. The method of claim 3, wherein the pain or discomfort is muscle or tendon condition related pain and the inversely linked neuro-receptor is selected from the group consisting of:

| Point of pain | Point of treatment |
|---|---|
| Knee Flexors | Elbow Flexors |
| Knee Extensors | Elbow Extensors |
| Knee Internal Rotators | Elbow External Rotators |
| Knee External Rotators | Elbow Internal Rotators |
| Wrist Flexors | Ankle Plantar Flexors |
| Wrist Extensors | Ankle Dorsiflexors |
| Wrist Medial Flexors | Ankle Pronators |
| Wrist Lateral Flexors | Ankle Supinators |
| Shoulder Flexors | Hip Extensors |
| Shoulder Extensors | Hip Flexors |
| Shoulder Abductors | Hip Abductors |
| Shoulder Adductors | Hip Adductors |
| Shoulder Internal Rotators | Hip Extrnal Rotators |
| Shoulder External Rotators | Hip External Rotators |
| Neck Flexors | Lumbar Flexors |
| Neck Extensors | Lumbar Extensors |
| Cervical Rotators (SCM) | Ipsilateral Illiopsoa Muscle Insertion |
| Supra Spinatus | Gluteus Minimus/Subclavicus |

8. A method of mechanical stimulation of an inversely linked dermatome for pain relief comprising:
identify a point of pain or discomfort on a patient's body;
selecting an inversely linked dermatome for the point of pain or discomfort;
select the appropriate mechanical nerve stimulation device for the pain or discomfort; and
administer mechanical nerve stimulation inversely and/or contralaterally to the inversely linked dermatome.

9. The method of claim 8, wherein the mechanical nerve stimulation device is selected from the group consisting of: a high frequency percussive instrument, a high velocity-low amplitude instrument, a laser; an electrical stimulator, a TENS machine, and combinations thereof.

10. The method of claim 8, wherein the pain or discomfort is nerve or motor control related pain and the inversely linked dermatome is selected from the group consisting of:

| Point of pain Dermatome | Point of treatment Dermatome |
|---|---|
| C1 | COCCYX |
| C2 | SI SACROILIAC NERVE |
| C3 | L5 |
| C4 | L4 |
| C5 | L3 |
| C6 | L2 |
| C7 | L1 |
| T1 | T12 |
| T2 | T11 |
| T3 | T10 |
| T4 | T9 |
| T5 | T8 |
| T6 | T7 |
| T7 | T6 |
| T8 | T5 |
| T9 | T4 |
| T10 | T3 |
| T11 | T2 |
| T12 | T1 |
| L1 | C7 |
| L2 | C6 |
| L3 | C5 |
| L4 | C4 |
| L5 | C3 |
| SI Sacroiliac Nerve | C2 |
| COCCYX Phalangeal | C1. |

11. The method of claim 8, wherein the pain or discomfort is an unspecified localized pain and the inversely linked dermatome is selected from the group consisting of:

| Point of pain Dermatome | Point of treatment Dermatome |
|---|---|
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |
| Acetabular Joint | Gleno-Humeral Joint |
| Anterior Iliac Spine | Acromioclavicular Joint |
| Posterior Superior Iliac Spine | Sterno-Clavicular Joint |
| Fibulo-Femoral Joint | Ulnar-Humeral Joint |
| Tibio-Femoral Joint | Radio-Humeral Joint |
| Inferior Patella | Olecranon Process |
| Tibio-Fibular Joint | Radio-Ulnar Joint |
| Point of pain | Point of treatment |
| T.M.J pain | Hip Joint |
| Gleno-Humeral Joint | Acetabular Joint |
| Acromioclavicular Joint | Anterior Iliac Spine |
| Sterno-Clavicular Joint | Posterior Superior Iliac Spine |
| Ulnar-Humeral Joint | Fibulo-Femoral Joint |
| Radio-Humeral Joint | Tibio-Femoral Joint |
| Olecranon Process | Inferior Patella |
| Radio-Ulnar Joint | Tibio-Fibular Joint |
| Hip Joint | T.M.J pain |

12. A method of mechanical stimulation of an inversely linked neuro-receptor for joint site discomfort related pain relief comprising:
identify a point of pain or discomfort on a patient's body;
selecting an inversely linked neuro-receptor for the point of the joint site discomfort related pain or discomfort;
select the appropriate mechanical nerve stimulation device for the joint site discomfort related pain or discomfort; and
administer mechanical nerve stimulation inversely and/or contralaterally to the inversely linked neuro-receptor.

13. The method of claim 12, wherein the mechanical nerve stimulation device is selected from the group consisting of: a high frequency percussive instrument, a high velocity-low amplitude instrument, a laser; an electrical stimulator, a TENS machine, and combinations thereof.

14. The method of claim 12, wherein the inversely linked neuro-receptor for joint site discomfort related pain is selected from the group consisting of:

| Point of pain Dermatome | Point of treatment Dermatome |
|---|---|
| Gleno-Humeral Joint | Acetabular Joint |
| Stemo-Clavicular Joint | Posterior Superior Illiac Spine |
| Ulnar-Humeral Joint | Fibulo-Femoral Joint |
| Radio-Humeral Joint | Tibia-Femoral Joint |
| Olcranon Process | Inferior Patella |
| Radio-Ulnar Joint | Tibia-Fibular Joint |
| Radial Site | Medial-Malleolus |
| Ulnar Site | Lateral-Malleolus |
| Scaphoid | Navicular |
| Lunate | Medial-Calcaneus |
| Triquetrum | Lateral Calcaneal |
| Hamate | Lateral Cuneiform |
| Trapezium | Intermedial Cuneiform |
| Trapezoid | Medial Cuneiform |
| Capitate | Cuboid |
| Meta-Carpo-Carpal Joint | Meta-Tarsal Joint |
| Meta-Carpo-Phalangeal | Meta-Tarso-Halux Phalangeal |
| Policis | Halux |
| Proximal Phalangeal | Pedal Proximal Phalangeal |

15. A method of mechanical stimulation of an inversely linked neuro-receptor for muscle or tendon condition related pain relief comprising:
identify a point of pain or discomfort on a patient's body;
selecting an inversely linked neuro-receptor for the point of pain or discomfort;
select the appropriate mechanical nerve stimulation device for the pain or discomfort; and
administer mechanical nerve stimulation inversely and/or contralaterally to the inversely linked neuro-receptor.

16. The method of claim 15, wherein the mechanical nerve stimulation device is selected from the group consisting of: a high frequency percussive instrument, a high velocity-low amplitude instrument, a laser; an electrical stimulator, a TENS machine, and combinations thereof.

17. The method of claim 15, wherein the inversely linked neuro-receptor for muscle or tendon condition related pain is selected from the group consisting of:

| Point of pain | Point of treatment |
|---|---|
| Knee Flexors | Elbow Flexors |
| Knee Extensors | Elbow Extensors |
| Knee Internal Rotators | Elbow External Rotators |
| Knee External Rotators | Elbow Internal Rotators |
| Wrist Flexors | Ankle Plantar Flexors |
| Wrist Extensors | Ankle Dorsiflexors |
| Wrist Medial Flexors | Ankle Pronators |
| Wrist Lateral Flexors | Ankle Supinators |
| Shoulder Flexors | Hip Extensors |
| Shoulder Extensors | Hip Flexors |
| Shoulder Abductors | Hip Abductors |
| Shoulder Adductors | Hip Adductors |
| Shoulder Internal Rotators | Hip Extrnal Rotators |
| Shoulder External Rotators | Hip External Rotators |
| Neck Flexors | Lumbar Flexors |
| Neck Extensors | Lumbar Extensors |
| Cervical Rotators (SCM) | Ipsilateral Illiopsoa Muscle Insertion |
| Supra Spinatus | Gluteus Minimus/Subclavicus |
